(19) United States
(12) Patent Application Publication
(10) Pub. No.: US 2024/0079650 A1
(43) Pub. Date: Mar. 7, 2024

(54) ADDITIVE FOR ELECTROLYTE, AND ELECTROLYTE FOR RECHARGEABLE LITHIUM BATTERY AND RECHARGEABLE LITHIUM BATTERY INCLUDING THE SAME

(71) Applicant: SAMSUNG SDI CO., LTD., Yongin-si (KR)

(72) Inventors: Hongryeol PARK, Yongin-si (KR); Hyunbong CHOI, Yongin-si (KR); Sangwoo PARK, Yongin-si (KR); Dahyun KIM, Yongin-si (KR); Sundae KIM, Yongin-si (KR); Yeji YANG, Yongin-si (KR); Olga TSAY, Yongin-si (KR); Pavel SHATUNOV, Yongin-si (KR); Sanghoon KIM, Yongin-si (KR)

(21) Appl. No.: 18/360,702

(22) Filed: Jul. 27, 2023

(30) Foreign Application Priority Data
Aug. 22, 2022 (KR) 10-2022-0104752
Jun. 29, 2023 (KR) 10-2023-0084415

(51) Int. Cl. H01M 10/0567; H01M 4/38; H01M 10/052; H01M 10/0569

(52) U.S. Cl. CPC H01M 10/0567; H01M 4/386; H01M 10/052; H01M 10/0569; H01M 2004/027

(57) ABSTRACT

An additive for an electrolyte, an electrolyte including the additive for a rechargeable lithium battery, and a rechargeable lithium battery including the electrolyte are provided. The additive may be represented by Chemical Formula 1, where at least one selected from A and B is a group represented by Chemical Formula A.

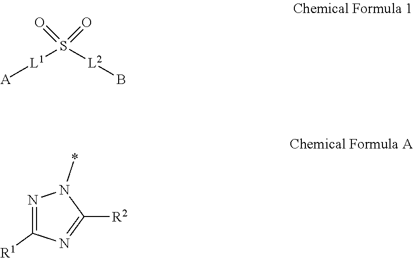

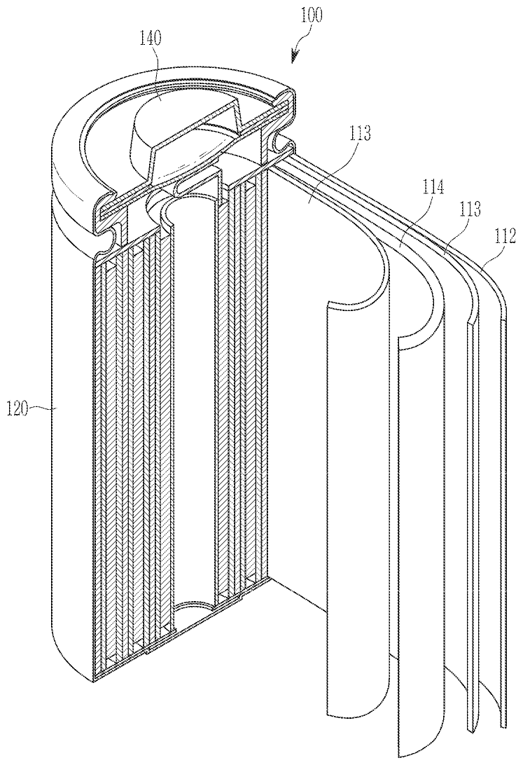

### CROSS-REFERENCE TO RELATED APPLICATION

This application claims priority to and the benefit of Korean Patent Application No. 10-2022-0104752 filed in the Korean Intellectual Property Office on Aug. 22, 2022, and Korean Patent Application No. 10-2023-0084415 filed in the Korean Intellectual Property Office on Jun. 29, 2023, the entire contents of which are incorporated herein by reference.

### BACKGROUND

### 1. Field

One or more embodiments of the present disclosure relate to an additive for an electrolyte, an electrolyte including the additive for a rechargeable lithium battery, and a rechargeable lithium battery including the electrolyte.

2. Description of the Related Art

A rechargeable lithium battery may be recharged and has three or more times energy density per unit weight as a lead storage battery, nickel-cadmium battery, nickel hydrogen battery, nickel zinc battery, and/or the like. It may be also charged at a high rate and thus, is commercially manufactured and utilized for a laptop, a cell phone, an electric tool, an electric bike, and/or the like. Research on improvement of the energy density of rechargeable lithium batteries has been actively made.

Such a rechargeable lithium battery is manufactured by injecting an electrolyte into a battery cell, which includes a positive electrode including a positive active material capable of intercalating/deintercalating lithium ions and a negative electrode including a negative active material capable of intercalating/deintercalating lithium ions.

The electrolyte serves as a medium for moving and transporting lithium ions between the negative electrode and the positive electrode. In general, an organic solvent in which a lithium salt is dissolved is utilized, and this electrolyte is an important factor in determining the stability and performance of a rechargeable lithium battery.

The electrolyte may include, for example, a mixed solvent of a high dielectric cyclic carbonate, such as propylene carbonate and ethylene carbonate, and a chain carbonate, such as diethyl carbonate, ethylmethyl carbonate, dimethyl carbonate, to which a lithium salt of LiPF₆, LiBF₄, lithium bis(fluorosulfonyl)imide (LiFSI), and/or the like is added. As the development of rechargeable lithium batteries in various fields is activated and progressed, the development of rechargeable lithium batteries with high output and high stability in a wide temperature range is becoming more important or highly desired. In terms of electrolyte, it is important to develop an optimal or suitable combination of an organic solvent and an additive capable of improving high output, long cycle-life, and high-temperature storage of the rechargeable lithium batteries, and suppressing swelling, capacity reduction, and resistance increase of the rechargeable lithium batteries.

### SUMMARY

One or more aspects of embodiments of the present disclosure are directed toward an additive for an electrolyte having excellent or suitable room temperature characteristics and high temperature characteristics.

One or more aspects of embodiments of the present disclosure are directed toward to an electrolyte for a rechargeable lithium battery including the additive.

One or more aspects of embodiments of the present disclosure are directed toward a rechargeable lithium battery including the electrolyte.

Additional aspects will be set forth in part in the description which follows and, in part, will be apparent from the description, or may be learned by practice of the presented embodiments of the disclosure.

According to one or more embodiments of the present disclosure, an additive for an electrolyte is provided and is represented by Chemical Formula 1.

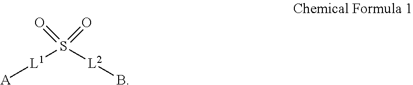

In Chemical Formula 1,

L¹ and L² may each independently be a single bond, a substituted or unsubstituted C1 to C5 alkylene group, a substituted or unsubstituted C2 to C5 alkenylene group, a substituted or unsubstituted C2 to C5 alkynylene group, or a substituted or unsubstituted C6 to C20 arylene group,

A and B may each independently be a substituted or unsubstituted C1 to C20 alkyl group, a substituted or unsubstituted C6 to C20 aryl group, or a substituted or unsubstituted C2 to C20 heteroaryl group, and

at least one selected from among A and B is a group represented by Chemical Formula A.

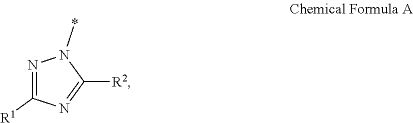

wherein, in Chemical Formula A,

R¹ and R² may each independently be hydrogen, a halogen, a substituted or unsubstituted C1 to C10 alkyl group, or a substituted or unsubstituted C3 to C10 cycloalkyl group.

In one or more embodiments, at least one selected from among L¹ and L² may be a substituted or unsubstituted C1 to C5 alkylene group.

L¹ and L² may each independently be a substituted or unsubstituted C1 to C5 alkylene group.

In one or more embodiments, at least one selected from among L¹ and L² may be a substituted or unsubstituted C2 to C5 alkylene group.

L¹ and L² may each independently be a substituted or unsubstituted C2 to C5 alkylene group.

In one or more embodiments, Chemical Formula 1 may be represented by Chemical Formula 1-1.

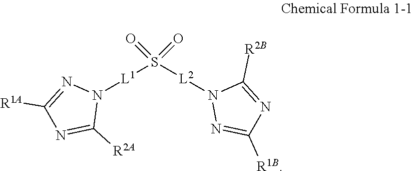

In Chemical Formula 1-1,

L¹ and L² may each independently be a substituted or unsubstituted C2 to C5 alkylene group,

R^{1A}, R^{1B}, R^{2A}, and R^{2B} may each independently be hydrogen, a halogen, a substituted or unsubstituted C1 to C10 alkyl group, or a substituted or unsubstituted C3 to C10 cycloalkyl group.

In one or more embodiments, the additive for an electrolyte represented by Chemical Formula 1 may be selected from among compounds of Group 1.

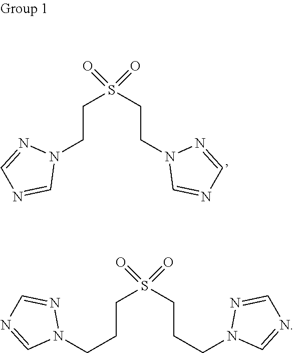 [SMILES: O=S(=O)(CCn1cncn1)CCn1cncn1 ; O=S(=O)(CCCn1cncn1)CCCn1cncn1]

According to one or more embodiments of the present disclosure, an electrolyte for a rechargeable lithium battery is provided and may include a non-aqueous organic solvent, a lithium salt, and the aforementioned additive for the electrolyte.

In some embodiments, the additive for the electrolyte may be included in an amount of about 0.01 to 5.0 parts by weight based on 100 parts by weight of the electrolyte for the rechargeable lithium battery.

In some embodiments, the additive for the electrolyte may be included in an amount of about 0.05 to 3.0 parts by weight based on 100 parts by weight of the electrolyte for the rechargeable lithium battery.

In some embodiments, the electrolyte for the rechargeable lithium battery may further include at least one other additive selected from among vinylene carbonate (VC), fluoroethylene carbonate (FEC), difluoroethylene carbonate, chloroethylene carbonate, dichloroethylene carbonate, bromoethylene carbonate, dibromoethylene carbonate, nitroethylene carbonate, cyanoethylene carbonate, vinylethylene carbonate (VEC), adiponitrile (AN), succinonitrile (SN), 1,3,6-hexane tricyanide (HTCN), propenesultone (PST), propanesultone (PS), lithium tetrafluoroborate (LiBF₄), lithium difluorophosphate (LiPO₂F₂), and 2-fluoro biphenyl (2-FBP).

According to one or more embodiments of the present disclosure, a rechargeable lithium battery is provided and may include a positive electrode including a positive active material; a negative electrode including a negative active material; and the aforementioned electrolyte for the rechargeable lithium battery.

The negative active material may include at least one selected from among graphite and Si composite.

In some embodiments, the Si composite may include a core including Si-based particles and an amorphous carbon coating layer.

In some embodiments, the Si-based particles may include at least one selected from among Si particles, a Si—C composite, SiOₓ (0<x≤2), and a Si alloy.

In some embodiments, the rechargeable lithium battery may operate at a high voltage of greater than or equal to about 4.3 V.

Thus, provided is a rechargeable lithium battery having improved cycle-life characteristics at room temperature and high temperature and excellent or suitable effect of suppressing an increase in resistance of the battery when left (e.g., stored) and utilized at a high temperature and suppressing gas generation.

### BRIEF DESCRIPTION OF THE DRAWINGS

The accompanying drawing is included to provide a further understanding of the present disclosure, and is incorporated in and constitutes a part of this specification. The drawing illustrates example embodiments of the present disclosure and, together with the description, serves to explain principles of present disclosure. In the drawings:

The drawing is a schematic view of a rechargeable lithium battery according to one or more embodiments of the present disclosure.

### DETAILED DESCRIPTION

The present disclosure may be modified in many alternate forms, and thus specific embodiments will be exemplified in the drawing and described in more detail. It should be understood, however, that it is not intended to limit the present disclosure to the particular forms disclosed, but rather, is intended to cover all modifications, equivalents, and alternatives falling within the spirit and scope of the present disclosure.

Hereinafter, embodiments of the present disclosure are described in more detail. However, these embodiments are example, the present disclosure is not limited thereto, and the present disclosure is defined by the scope of claims.

As utilized herein, when a definition is not otherwise provided, “substituted” may refer to replacement of hydrogen of a compound by a substituent selected from among deuterium, a halogen (F, Br, CI, or I), a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 fluoroalkyl group, a cyano group, and combination thereof.

As utilized herein, when a definition is not otherwise provided, “substituted” may refer to replacement of at least one hydrogen of a substituent or a compound by deuterium, a halogen, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C10 fluoroalkyl group, or a cyano group. In some embodiments, “substituted” may refer to replacement of at least one hydrogen of a substituent or a compound by deuterium, a halogen, a C1 to C20 alkyl group, a C6 to C30 aryl group, a C1 to C10 fluoroalkyl group, or a cyano group. In some embodiments, “substituted” may refer to replacement of at least one hydrogen of a substituent or a compound by deuterium, a halogen, a C1 to C5 alkyl group, a C6 to C18 aryl group, a C1 to C5 fluoroalkyl group, or a cyano group. In some embodiments, “substituted” may refer to replacement of at least one hydrogen of a substituent or a compound by deuterium, a cyano group, a halogen, a methyl group, an ethyl group, a propyl group, a butyl group, a phenyl group, a biphenyl group, a terphenyl group, a trifluoromethyl group, or a naphthyl group.

A rechargeable lithium battery may be classified into a lithium ion battery, a lithium ion polymer battery, or a lithium polymer battery depending on kinds or types of a separator and an electrolyte. It also may be classified to be cylindrical, prismatic, coin-type or kind, pouch-type or kind, and/or the like depending on shapes. In some embodiments, it may be bulk type or kind or thin film type or kind depending on sizes. Structures and manufacturing methods for lithium ion batteries pertaining to the present disclosure are suitably established in the art.

Herein, a cylindrical rechargeable lithium battery will be exemplarily described as an example of the rechargeable lithium battery. The drawing schematically shows the structure of a rechargeable lithium battery according to one or more embodiments of the present disclosure. Referring to the drawing, in one or more embodiments, a rechargeable lithium battery 100 may include a battery cell including a positive electrode 114, a negative electrode 112 facing the positive electrode 114, a separator 113 between the positive electrode 114 and the negative electrode 112, an electrolyte impregnating the positive electrode 114, the negative electrode 112, and the separator 113, a battery case 120 housing the battery cell, and a sealing member 140 sealing the battery case 120.

Hereinafter, an additive for an electrolyte according to one or more embodiments of the present disclosure will be described.

In one or more embodiments, the additive may be represented by Chemical Formula 1.

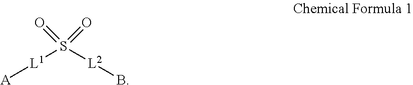

In Chemical Formula 1,

L¹ and L² may each independently be a single bond, a substituted or unsubstituted C1 to C5 alkylene group, a substituted or unsubstituted C2 to C5 alkenylene group, a substituted or unsubstituted C2 to C5 alkynylene group, or a substituted or unsubstituted C6 to C20 arylene group,

A and B may each independently be a substituted or unsubstituted C1 to C20 alkyl group, a substituted or unsubstituted C6 to C20 aryl group, or a substituted or unsubstituted C2 to C20 heteroaryl group, and

at least one selected from among A and B is a group represented by Chemical Formula A,

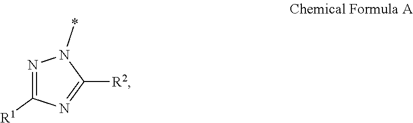

wherein, in Chemical Formula A,

R¹ and R² may each independently be hydrogen, a halogen, a substituted or unsubstituted C1 to C10 alkyl group, or a substituted or unsubstituted C3 to C10 cycloalkyl group.

In a rechargeable lithium battery, a non-aqueous electrolyte may be decomposed during the initial charge and discharge to form a film having passivation ability on the surfaces of positive and negative electrodes. The formed film may improve storage characteristics at a high temperature, but may be deteriorated by acid such as HF and PF₅ produced by thermal decomposition of lithium salts (LiPF₆ and/or the like) that are widely utilized in lithium ion batteries. This acid attack elutes transition metal elements from the positive electrode, which increases sheet resistance of the electrode due to structural changes of the surface and reduces theoretical capacity due to the loss of the transition metal elements, which are redox centers, thereby deteriorating expression capacity of the lithium ion battery. In addition, these eluted transition metal ions are electrodeposited on the negative electrode reacting in a strong reduction potential and thus may not only consume electrons but also destroy the film during the electrodeposition and expose the surface of the negative electrode, which additionally causes the decomposition reaction of the electrolyte. As a result, as the resistance of the negative electrode increases, and irreversible capacity of the negative electrode increases, there is a problem of continuously deteriorating capacity of a battery cell. In the present disclosure, a triazole group and a sulfone group of the additive represented by Chemical Formula 1 provide a lone pair of electrons to capture PF₅ and stabilize LiPF₆ salt, thereby removing the acid produced due to the decomposition of the lithium salt.

In some embodiments, the sulfone group included in Chemical Formula 1 forms a film on the surface of the positive electrode to suppress or reduce decomposition of the positive active material, thereby suppressing or reducing gas generation and elution of transition metals due to the decomposition of the positive electrode material.

In some embodiments, the additive represented by Chemical Formula 1 may strengthen the solid electrolyte interphase (SEI) film on the surface of the positive electrode while preventing or reducing deterioration of the SEI film or elution of transition metals from the positive electrode during high-temperature storage.

In some embodiments, at least one selected from among L¹ and L² may be a substituted or unsubstituted C1 to C5 alkylene group.

In some embodiments, L¹ and L² may each independently be a substituted or unsubstituted C1 to C5 alkylene group.

In some embodiments, at least one selected from among L¹ and L² may be a substituted or unsubstituted C2 to C5 alkylene group.

In some embodiments, L¹ and L² may each independently be a substituted or unsubstituted C2 to C5 alkylene group.

In one or more embodiments, Chemical Formula 1 may be represented by Chemical Formula 1-1.

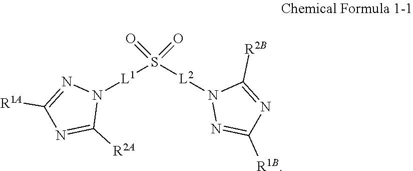

In Chemical Formula 1-1,

L¹ and L² may each independently be a substituted or unsubstituted C2 to C5 alkylene group, and

R^{1A}, R^{1B}, R^{2A}, and R^{2B} may each independently be hydrogen, a halogen, a substituted or unsubstituted C1 to C10 alkyl group, or a substituted or unsubstituted C3 to C10 cycloalkyl group.

In one or more embodiments, the additive for the electrolyte may be selected from among the compounds of Group 1.

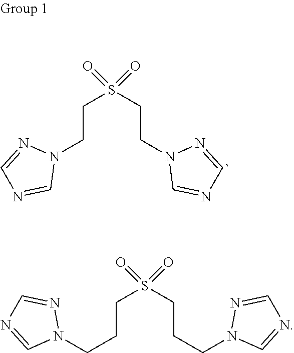 [SMILES: O=S(=O)(CCn1cncn1)CCn1cncn1 ; O=S(=O)(CCCn1cncn1)CCCn1cncn1]

In one or more embodiments, the electrolyte for a rechargeable lithium battery may include a non-aqueous organic solvent, a lithium salt, and the aforementioned additive for the electrolyte.

In some embodiments, the additive may be included in an amount of about 0.01 to 5.0 parts by weight based on 100 parts by weight of the electrolyte for a rechargeable lithium battery.

For example, in some embodiments, the additive may be included in an amount of about 0.01 to 4.0 parts by weight, about 0.01 to 3.0 parts by weight, about 0.03 to 3.0 parts by weight, or about 0.05 to 3.0 parts by weight based on 100 parts by weight of the electrolyte for a rechargeable lithium battery.

When the content (e.g., amount) range of the additive in the electrolyte is within the range described above, it is possible to implement a rechargeable lithium battery having improved cycle-life characteristics and output characteristics by preventing or reducing an increase in resistance at high temperatures.

In one or more embodiments, the electrolyte for a rechargeable lithium battery may further include at least one other additive selected from among vinylene carbonate (VC), fluoroethylene carbonate (FEC), difluoroethylene carbonate, chloroethylene carbonate, dichloroethylene carbonate, bromoethylene carbonate, dibromoethylene carbonate, nitroethylene carbonate, cyanoethylene carbonate, vinylethylene carbonate (VEC), adiponitrile (AN), succinonitrile (SN), 1,3,6-hexane tricyanide (HTCN), propenesultone (PST), propanesultone (PS), lithium tetrafluoroborate (LiBF₄), lithium difluorophosphate (LiPO₂F₂), and 2-fluoro biphenyl (2-FBP).

By further including the aforementioned other additives in the electrolyte, the cycle-life of the rechargeable lithium battery may be further improved, and/or gases generated from the positive electrode and the negative electrode may be effectively controlled or reduced during high-temperature storage.

In one or more embodiments, the other additives may be included in an amount of about 0.2 to 20 parts by weight, about 0.2 to 15 parts by weight, or about 0.2 to 10 parts by weight, based on 100 parts by weight of the electrolyte for a rechargeable lithium battery.

When the content (e.g., amount) of other additives in the electrolyte is within the range described above, an increase in film resistance may be minimized or reduced, thereby contributing to the improvement of battery performance.

The non-aqueous organic solvent serves as a medium for transporting ions taking part in the electrochemical reaction of a battery.

The non-aqueous organic solvent may be a carbonate-based, ester-based, ether-based, ketone-based, alcohol-based, or aprotic solvent.

In some embodiments, the carbonate-based solvent may include dimethyl carbonate (DMC), diethyl carbonate (DEC), dipropyl carbonate (DPC), methylpropyl carbonate (MPC), ethylpropyl carbonate (EPC), ethylmethyl carbonate (EMC), ethylene carbonate (EC), propylene carbonate (PC), butylene carbonate (BC), and/or the like. In some embodiments, the ester-based solvent may include methyl acetate, ethyl acetate, n-propyl acetate, t-butyl acetate, methylpropionate, ethylpropionate, propylpropionate, decanolide, mevalonolactone, caprolactone, and/or the like. In some embodiments, the ether-based solvent may include dibutyl ether, tetraglyme, diglyme, dimethoxyethane, 2-methyltetrahydrofuran, tetrahydrofuran, and/or the like. In some embodiments, the ketone-based solvent may include cyclohexanone, and/or the like. In some embodiments, The alcohol-based solvent may include ethanol, isopropyl alcohol, and/or the like. In some embodiments, the aprotic solvent may include nitriles such as R¹—CN (wherein R¹ is a hydrocarbon group having a C2 to C20 linear, branched, or cyclic structure and may include a double bond, an aromatic ring, and/or an ether bond), and/or the like, dioxolanes such as 1,3-dioxolane, and/or the like, sulfolanes, and/or the like.

The non-aqueous organic solvent may be utilized alone or in a mixture. When the non-aqueous organic solvent is utilized in a mixture, the mixture ratio may be controlled or selected in accordance with a desirable battery performance.

In some embodiments, the carbonate-based solvent may be prepared by mixing a cyclic carbonate and a chain carbonate. When the cyclic carbonate and chain carbonate are mixed together in a volume ratio of about 1:1 to about 9:1, an electrolyte performance may be improved.

In one or more embodiments of the present disclosure, the non-aqueous organic solvent may include a cyclic carbonate and a chain carbonate in a volume ratio of about 2:8 to about 5:5. In some embodiments, the cyclic carbonate and the chain carbonate may be included in a volume ratio of about 2:8 to about 4:6.

In some embodiments, the cyclic carbonate and the chain carbonate may be included in a volume ratio of about 2:8 to about 3:7.

In one or more embodiments, the non-aqueous organic solvent may further include an aromatic hydrocarbon-based organic solvent in addition to the carbonate-based solvent. In some embodiments, the carbonate-based solvent and the aromatic hydrocarbon-based organic solvent may be mixed in a volume ratio of about 1:1 to about 30:1.

In one or more embodiments, the aromatic hydrocarbon-based organic solvent may be an aromatic hydrocarbon-based compound of Chemical Formula 3.

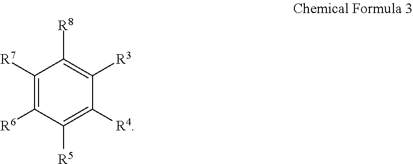

In Chemical Formula 3, R³ to R⁸ may be the same or different and are each independently selected from among hydrogen, a halogen, a C1 to C10 alkyl group, a C1 to C10 haloalkyl group, and a combination thereof.

Non-limiting examples of the aromatic hydrocarbon-based organic solvent may be selected from among benzene, fluorobenzene, 1,2-difluorobenzene, 1,3-difluorobenzene, 1,4-difluorobenzene, 1,2,3-trifluorobenzene, 1,2,4-trifluorobenzene, chlorobenzene, 1,2-dichlorobenzene, 1,3-dichlorobenzene, 1,4-dichlorobenzene, 1,2,3-trichlorobenzene, 1,2,4-trichlorobenzene, iodobenzene, 1,2-diiodobenzene, 1,3-diiodobenzene, 1,4-diiodobenzene, 1,2,3-triiodobenzene, 1,2,4-triiodobenzene, toluene, fluorotoluene, 2,3-difluorotoluene, 2,4-difluorotoluene, 2,5-difluorotoluene, 2,3,4-trifluorotoluene, 2,3,5-trifluorotoluene, chlorotoluene, 2,3-dichlorotoluene, 2,4-dichlorotoluene, 2,5-dichlorotoluene, 2,3,4-trichlorotoluene, 2,3,5-trichlorotoluene, iodotoluene, 2,3-diiodotoluene, 2,4-diiodotoluene, 2,5-diiodotoluene, 2,3,4-triiodotoluene, 2,3,5-triiodotoluene, xylene, and a combination thereof.

The lithium salt dissolved in the non-aqueous organic solvent supplies lithium ions in a rechargeable lithium battery, enables a basic operation of the rechargeable lithium battery, and improves transportation of the lithium ions between positive and negative electrodes. Non-limiting examples of the lithium salt may include at least one selected from among LiPF₆, LiBF₄, LiSbF₆, LiAsF₆, Li(FSO₂)₂N (lithium bis(fluorosulfonyl)imide: LiFSI), LiC₄F₉SO₃, LiClO₄, LiAlO₂, LiAlCl₄, LiPO₂F₂, LiN(CₓF₂ₓ₊₁SO₂)(C_{y}F_{2y+1}SO₂), (wherein, x and y are an integer in a range of 1 to 20), LiCl, LiI, LiB(C₂O₄)₂ (lithium bis(oxalato) borate: LiBOB), LiDFOB (lithium difluoro(oxalato)borate), and Li[PF₂(C₂O₄)_{2]} (lithium difluoro(bisoxalato) phosphate). In one or more embodiments, the lithium salt may be utilized in a concentration in a range of about 0.1 M to about 2.0 M. When the lithium salt is included at the above concentration range, the electrolyte may have excellent or suitable performance and lithium ion mobility due to optimal or suitable electrolyte conductivity and viscosity.

In one or more embodiments, a rechargeable lithium battery is provided to include a positive electrode including a positive active material; a negative electrode including a negative active material; and the aforementioned electrolyte.

The positive electrode may include a positive electrode current collector and a positive active material layer on the positive electrode current collector, and the positive active material layer may include a positive active material.

The positive active material may include lithiated intercalation compounds that reversibly intercalate and deintercalate lithium ions.

For example, in one or more embodiments, at least one composite oxide of a metal selected from among cobalt, manganese, nickel, iron, and a combination thereof, and lithium may be utilized.

Of course, the composite oxide in which a portion of the metal is substituted with other metal may also be utilized. In some embodiments, a phosphoric acid compound of the composite oxide, for example, at least one selected from among LiNiPO₄, LiFePO₄, LiCoPO₄, and LiMnPO₄, may be utilized. In some embodiments, one having a coating layer on the surface of the oxide may be utilized, or a mixture of the composite oxide and the composite oxide having a coating layer may be utilized. The coating layer may include at least one coating element compound selected from among an oxide of a coating element, a hydroxide of a coating element, an oxyhydroxide of a coating element, an oxycarbonate of a coating element, and a hydroxy carbonate of a coating element. The coating element compound for the coating layer may be amorphous or crystalline. The coating element included in the coating layer may include magnesium (Mg), aluminum (Al), cobalt (Co), potassium (K), sodium (Na), calcium (Ca), silicon (Si), titanium (Ti), vanadium (V), tin (Sn), germanium (Ge), gallium (Ga), boron (B), arsenic (As), zirconium (Zr), or a mixture thereof. The coating layer may be disposed in a method having no adverse influence on properties of a positive active material by utilizing these elements in the compound. For example, the method may include any coating method (e.g., spray coating, dipping, etc.), but will not be illustrated in more detail because it is well suitable and established to those skilled in the related field.

In one or more embodiments, the positive active material may be, for example, at least one selected from lithium composite oxides represented by Chemical Formula 4.

LiₓM¹_{y}M²_{z}M³_{1-y-z}O_{2±a}Xₐ Chemical Formula 4

In Chemical Formula 4,

0.5≤x≤1.8, 0≤a≤0.1, 0≤y≤1, 0≤z≤1, 0<y+z≤1, M¹, M², and M³ may each independently be one or more elements selected from among nickel (Ni), cobalt (Co), manganese (Mn), aluminum (Al), boron (B), barium (Ba), calcium (Ca), cerium (Ce), chromium (Cr), iron (Fe), molybdenum (Mo), niobium (Nb), silicon (Si), strontium (Sr), magnesium (Mg), titanium (Ti), vanadium (V), tungsten (W), zirconium (Zr), and lanthanum (La), and a combination thereof, and X may include one or more elements selected from among fluorine (F), sulfur (S), phosphorus (P), and chlorine (Cl).

In one or more embodiments, the positive active material may be at least one selected from among LiCoO₂, LiNiO₂, LiMnO₂, LiMn₂O₄, LiNiₐMn_{b}Co_{c}O₂ (a+b+c=1), LiNiₐMn_{b}Co_{c}Al_{d}O₂ (a+b+c+d=1), and LiNiₑCo_{f}Al_{g}O₂ (e+f+g=1).

In Chemical Formula 4, 0.8≤y≤1, 0≤z≤0.2, and M¹ may be Ni.

For example, in some embodiments, the positive active material selected from among LiNi_{b}Mn_{c}Co_{d}O₂ (b+c+d=1), LiNi_{b}Mn_{c}Co_{d}AlₑO₂ (b+c+d+e=1), and LiNi_{b}Co_{d}AlₑO₂ (b+d+e=1) may be a high Ni-based positive active material.

For example, in embodiments of LiNi_{b}Mn_{c}Co_{d}O₂ (b+c+d=1) and LiNi_{b}Mn_{c}Co_{d}AlₑO₂ (b+c+d+e=1), a nickel content (e.g., amount) may be greater than or equal to about 60% (b≥0.6), and in some embodiments, greater than or equal to about 80% (b≥0.8).

For example, in embodiments of LiNi_{b}Co_{d}AlₑO₂ (b+d+e=1), a nickel content (e.g., amount) may be greater than or equal to about 60% (b≥0.6), and in some embodiments, greater than or equal to about 80% (b≥0.8).

In one or more embodiments, the positive active material may be included in an amount of about 90 wt % to about 98 wt % based on the total weight of the positive active material layer.

In one or more embodiments, a conductive material and a binder may be included in an amount of about 1 wt % to about 5 wt % based on the total weight of the positive active material layer.

The conductive material is included to provide electrode conductivity. Any electrically conductive material may be utilized as a conductive material unless it causes a chemical change. Non-limiting examples of the conductive material may include a carbon-based material such as natural graphite, artificial graphite, carbon black, acetylene black, ketjen black, a carbon fiber, and/or the like; a metal-based material of a metal powder or a metal fiber including copper, nickel, aluminum, silver, and/or the like; a conductive polymer such as a polyphenylene derivative; or a mixture thereof.

The binder may improve binding properties of positive active material particles with one another and with a current collector. Non-limiting examples thereof may be polyvinyl alcohol, carboxylmethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and/or the like, but embodiments of the present disclosure are not limited thereto.

The positive electrode current collector may utilize Al, but embodiments of the present disclosure are not limited thereto.

The negative electrode may include a negative electrode current collector and a negative active material layer including a negative active material formed on the negative electrode current collector.

The negative active material may include a material that reversibly intercalates/deintercalates lithium ions, a lithium metal, a lithium metal alloy, a material being capable of doping/dedoping lithium, or transition metal oxide.

The material that reversibly intercalates/deintercalates lithium ions may include a carbon material. The carbon material may be any generally utilized carbon-based negative active material in a rechargeable lithium battery. Non-limiting examples thereof may be crystalline carbon, amorphous carbon, or a mixture thereof. The crystalline carbon may be non-shaped (e.g., irregularly shaped), sheet, flake, or spherical, or fiber shaped natural graphite or artificial graphite. The amorphous carbon may be a soft carbon, a hard carbon, a mesophase pitch carbonization product, calcined coke, and/or the like.

The lithium metal alloy may include an alloy of lithium and a metal selected from among sodium (Na), potassium (K), rubidium (Rb), cesium (Cs), francium (Fr), beryllium (Be), magnesium (Mg), calcium (Ca), strontium (Sr), silicon (Si), antimony (Sb), lead (Pb), indium (In), zinc (Zn), barium (Ba), radium (Ra), germanium (Ge), aluminum (Al), and tin (Sn).

The material being capable of doping/dedoping lithium may be Si, Si—C composite, SiOₓ (0<x<2), a Si-Q alloy (wherein Q is an element selected from an alkali metal, an alkaline-earth metal, a Group 13 element, a Group 14 element except Si, a Group 15 element, a Group 16 element, a transition metal, a rare earth element, and a combination thereof), Sn, SnO₂, a Sn—R¹¹ alloy (wherein R¹¹ is an element selected from an alkali metal, an alkaline-earth metal, a Group 13 element, a Group 14 element except Sn, a Group 15 element, a Group 16 element, a transition metal, a rare earth element, and a combination thereof), and/or the like. In some embodiments, at least one selected from these materials may be mixed with SiO₂.

The elements Q and R¹¹ may be selected from among magnesium (Mg), calcium (Ca), strontium (Sr), barium (Ba), radium (Ra), scandium (Sc), ytterbium (Y), titanium (Ti), zirconium (Zr), hafnium (Hf), rutherfordium (Rf), vanadium (V), niobium (Nb), tantalum (Ta), dubnium (Db), chromium (Cr), molybdenum (Mo), tungsten (W), seaborgium (Sg), technetium (Tc), rhenium (Re), bohrium (Bh), iron (Fe), lead (Pb), ruthenium (Ru), osmium (Os), hassium (Hs), rhodium (Rh), iridium (Ir), palladium (Pd), platinum (Pt), copper (Cu), silver (Ag), gold (Au), zinc (Zn), cadmium (Cd), boron (B), aluminum (Al), gallium (Ga), tin (Sn), indium (In), thallium (Tl), germanium (Ge), phosphorus (P), arsenic (As), antimony (Sb), bismuth (Bi), sulfur (S), selenium (Se), tellurium (Te), polonium (Po), and a combination thereof.

The transition metal oxide may be vanadium oxide, lithium vanadium oxide, or lithium titanium oxide.

In one or more embodiments, the negative active material may include at least one selected from among graphite and a Si composite.

The Si composite may include a core including Si-based particles and an amorphous carbon coating layer, and for example, in some embodiments, the Si-based particles may include at least one selected from among Si particles, a Si—C composite, SiOₓ (0<x≤2), and a Si alloy.

In some embodiments, the Si composite may include a void in the center of the core including the Si-based particles, a radius of the core corresponds to 30% to 50% of the radius of the Si composite, an average particle diameter of the Si composite may be about 5 μm to 20 μm, and an average particle diameter of the Si-based particles may be about 10 nm to about 200 nm.

In the present disclosure, the average particle diameter may be a particle size (D50) at 50% by volume in a cumulative size-distribution curve.

When the average particle diameter of the Si-based particle is within the above range, volume expansion occurring during charging and discharging may be suppressed or reduced, and a break in a conductive path due to particle crushing during charging and discharging may be prevented or reduced.

In some embodiments, the core including the Si-based particles may further include amorphous carbon, and the central portion of the Si composite does not include amorphous carbon, and the amorphous carbon may exist only on the surface portion of the Si composite.

In embodiments of the present disclosure, the surface portion may refer to a region from the outermost surface of the central portion to the outermost surface of the Si composite.

In some embodiments, the Si-based particles are substantially uniformly included in the Si composite as a whole, that is, Si-based particles may be present in a substantially uniform concentration in the center portion and the surface portion of the Si composite.

The amorphous carbon may be soft carbon, hard carbon, a mesophase pitch carbonized product, calcined coke, or a combination thereof.

For example, in some embodiments, the Si—C composite may include Si particles and crystalline carbon.

The Si particles may be included in an amount of about 1 wt % to about 60 wt %, for example, in some embodiments, about 3 wt % to about 60 wt %, based on the total weight of the Si—C composite.

The crystalline carbon may be, for example, graphite, such as natural graphite, artificial graphite, or a combination thereof.

The average particle diameter of the crystalline carbon may be about 5 μm to about 30 μm.

When the negative active material includes both (e.g., simultaneously) graphite and Si composite, the graphite and Si composite may be included in the form of a mixture. For example, in some embodiments, the graphite and Si composite may be included in a weight ratio of about 99:1 to about 50:50.

In one or more embodiments, the graphite and Si composite may be included in a weight ratio of about 97:3 to about 80:20, or about 95:5 to about 80:20.

The amorphous carbon precursor may include a coal-based pitch, mesophase pitch, petroleum-based pitch, coal-based oil, petroleum-based heavy oil, and/or a polymer resin such as a phenol resin, a furan resin, and/or a polyimide resin.

In one or more embodiments, in the negative active material layer, the negative active material may be included in an amount of about 95 wt % to about 99 wt % based on the total weight of the negative active material layer.

In one or more embodiments of the present disclosure, the negative active material layer may include a binder, and optionally a conductive material. In the negative active material layer, a content (e.g., amount) of the binder may be about 1 wt % to about 5 wt % based on a total weight of the negative active material layer. When the negative active material layer includes a conductive material, the negative active material layer may include about 90 wt % to about 98 wt % of the negative active material, about 1 wt % to about 5 wt % of the binder, and about 1 wt % to about 5 wt % of the conductive material.

The binder may improve binding properties of negative active material particles with one another and with a current collector. The binder may include a non-water-soluble binder, a water-soluble binder, or a combination thereof.

The non-water-soluble binder may be selected from among polyvinylchloride, carboxylated polyvinylchloride, polyvinylfluoride, polyurethane, polytetrafluoroethylene, polyvinylidene fluoride, polyethylene, polypropylene, polyamideimide, polyimide, and a combination thereof.

The water-soluble binder may be a rubber-based binder or a polymer resin binder. The rubber-based binder may be selected from among a styrene-butadiene rubber, an acrylated styrene-butadiene rubber (SBR), an acrylonitrile-butadiene rubber, an acrylic rubber, a butyl rubber, a fluorine rubber, and a combination thereof. The polymer resin binder may be selected from among polytetrafluoroethylene, an ethylenepropylene copolymer, polyethyleneoxide, polyvinylpyrrolidone, polyepichlorohydrin, polyphosphazene, polyacrylonitrile, polystyrene, an ethylenepropylenediene copolymer, polyvinylpyridine, chlorosulfonated polyethylene, latex, a polyester resin, an acrylic resin, a phenolic resin, an epoxy resin, polyvinyl alcohol, and a combination thereof.

When the water-soluble binder is utilized as a negative electrode binder, a cellulose-based compound may be further utilized to provide viscosity as a thickener. The cellulose-based compound may include one or more selected from among carboxymethyl cellulose, hydroxypropylmethyl cellulose, methyl cellulose, and alkali metal salts thereof. The alkali metal may be Na, K, or Li. In some embodiments, such a thickener may be included in an amount of about 0.1 to about 3 parts by weight based on 100 parts by weight of the negative active material.

The conductive material may be included to provide electrode conductivity. Any electrically conductive material may be utilized as a conductive material unless it causes a chemical change. Non-limiting examples of the conductive material may include a carbon-based material such as natural graphite, artificial graphite, carbon black, acetylene black, ketjen black, a carbon fiber, and/or the like; a metal-based material of a metal powder or a metal fiber including copper, nickel, aluminum, silver, and/or the like; a conductive polymer such as a polyphenylene derivative; or a mixture thereof.

The negative electrode current collector may include one selected from among a copper foil, a nickel foil, a stainless steel foil, a titanium foil, a nickel foam, a copper foam, a polymer substrate coated with a conductive metal, and a combination thereof.

In one or more embodiments, the rechargeable lithium battery may further include a separator between the negative electrode and the positive electrode, depending on a type or kind of the rechargeable lithium battery. Such a separator may be a porous substrate, or a composite porous substrate.

The porous substrate may be a substrate including pores, through which lithium ions can move and pass. The porous substrate may include, for example, polyethylene, polypropylene, polyvinylidene fluoride, or a multilayer film of two or more layers thereof. In some embodiments, a mixed multilayer film such as a polyethylene/polypropylene double-layered separator, a polyethylene/polypropylene/polyethylene triple-layered separator, a polypropylene/polyethylene/polypropylene triple-layered separator, and/or the like may be utilized.

The composite porous substrate may have a form including a porous substrate and a functional layer on the porous substrate. The functional layer may be, for example, at least one selected from among a heat-resistant layer and an adhesive layer from the viewpoint of enabling additional functions to be added. For example, in some embodiments, the heat-resistant layer may include a heat-resistant resin and optionally a filler.

In some embodiments, the adhesive layer may include an adhesive resin and optionally a filler.

The filler may be an organic filler or an inorganic filler.

Hereinafter, examples of the present disclosure and comparative examples are described. These examples, however, are not in any sense to be interpreted as limiting the scope of the present disclosure.

### SYNTHESIS EXAMPLE: SYNTHESIS OF ADDITIVES

### Synthesis Example 1: Synthesis of Chemical Formula 1a

After preparing a solution by mixing 1H-1,2,4-triazole (2 mmol) with acetone and sufficiently stirring the mixture with sodium hydrogen carbonate (3 mmol), divinyl sulfone (1.1 mmol) was mixed with 30 mL of acetone and then, added dropwise to the solution for 30 minutes. Subsequently, the mixture was stirred at room temperature (25° C.) for 4 hours, and precipitates were filtered therefrom. The filtered solution was recrystallized, obtaining a compound of Chemical Formula 1a.

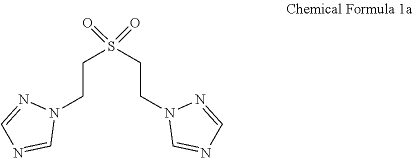 [SMILES: O=S(=O)(CCn1cncn1)CCn1cncn1]

### Synthesis Example 2: Synthesis of Chemical Formula 1b

A compound of Chemical Formula 1b was obtained in substantially the same manner as in Synthesis Example 1 except that 3-(allylsulfonyl)prop-1-ene was utilized instead of the divinyl sulfone.

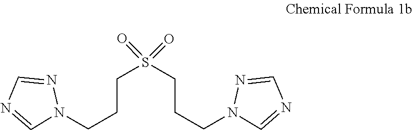 [SMILES: O=S(=O)(CCCn1cncn1)CCCn1cncn1]

### Synthesis Example 3: Synthesis of Chemical Formula 1c

1H-1,2,4-triazole was mixed with sodium hydroxide dissolved in water and then, heated at 200° C. for 40 minutes under 8 Pa. Subsequently, sulfuryl fluoride was mixed therewith and then, treated for 18 minutes under 67 kPa, obtaining a compound of Chemical Formula 1c.

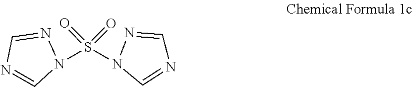 [SMILES: O=S(=O)(n1cncn1)n1cncn1]

### Comparative Synthesis Example 1: Synthesis of Chemical Formula C3

A compound of Chemical Formula C3 was obtained in substantially the same manner as in Synthesis Example 1 except that pyrrole was utilized instead of the 1H-1,2,4-triazole.

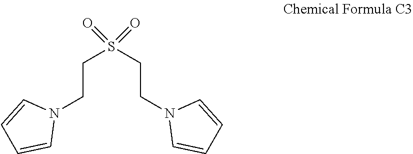 [SMILES: O=S(=O)(CCn1cccc1)CCn1cccc1]

### Comparative Synthesis Example 2: Synthesis of Chemical Formula C4

A compound of Chemical Formula C4 was obtained in substantially the same manner as in Synthesis Example 3 except that 4H-1,2,4-triazole was utilized instead of the 1H-1,2,4-triazole.

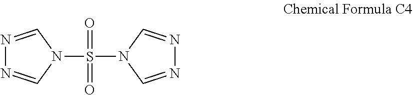 [SMILES: O=S(=O)(n1cnnc1)n1cnnc1]

Comparative Synthesis Example 3: Synthesis of Chemical Formula C5

A compound of Chemical Formula C5 was obtained in substantially the same manner as in Synthesis Example 3 except that 1H-imidazole was utilized instead of the 1H-1,2,4-triazole.

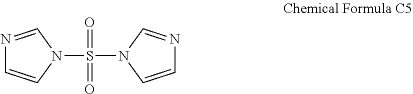 [SMILES: O=S(=O)(n1ccnc1)n1ccnc1]

### EXAMPLE: MANUFACTURE OF RECHARGEABLE LITHIUM BATTERY CELL

### Example 1

Positive active material slurry was prepared by utilizing LiNi_{0.88}Co_{0.7}Al_{0.06}O₂ as a positive active material, polyvinylidene fluoride as a binder, and acetylene black as a conductive material in a weight ratio of 96:2:2, and dispersing the mixture in N-methyl pyrrolidone.

The positive active material slurry was coated on a 14 μm-thick Al foil, dried at 110° C., and pressed to manufacture a positive electrode.

A mixture of artificial graphite and Si composite in a weight ratio of 93:7 as a negative active material, a styrene-butadiene rubber binder as a binder, and carboxymethyl cellulose as a thickener were mixed in a weight ratio of 97:1:2, respectively, and dispersed in distilled water to prepare a negative active material slurry.

The Si composite has a core including artificial graphite and silicon particles, and a coal-based pitch coated on the surface of the core.

The negative active material slurry was coated on a 10 μm-thick Cu foil, dried at 100° C., and pressed to manufacture a negative electrode.

An electrode assembly was manufactured by assembling the manufactured positive and negative electrodes, and a separator made of polyethylene having a thickness of 25 μm, and an electrolyte was injected to prepare a rechargeable lithium battery cell.

The electrolyte has a following composition.

### Composition of Electrolyte

Lithium salt: 1.15 M LiPF₆

non-aqueous organic solvent:ethylene carbonate (EC):ethylmethyl carbonate (EMC):dimethyl carbonate (DMC) (EC:EMC:DMC=a volume ratio of 20:10:70)

Additive: 0.5 parts by weight of the compound represented by Chemical Formula 1a, 10 parts by weight of fluoroethylene carbonate (FEC), and 0.5 parts by weight of succinonitrile (SN)

(in the composition of the electrolyte, “parts by weight” refers to a relative weight of the additive based on 100 weight of the total (lithium salt non-aqueous organic solvent) of the electrolyte excluding the additive.)

### Comparative Example 1

A rechargeable lithium battery cell was manufactured in substantially the same manner as in Example 1 except that an additive was not utilized.

### Examples 2 and 3 and Comparative Examples 2 to 6

Each rechargeable lithium battery cell was manufactured in substantially the same manner as in Example 1 except that the compound of Chemical Formula 1b (Example 2), the compound of Chemical Formula 1c (Example 3), the compound of Chemical Formula C1 (Wako Pure Chemical Industries, Ltd.) (Comparative Example 2), the compound of Chemical Formula C2 (Sigma-Aldrich Co., Ltd.) (Comparative Example 3), and the compounds of Chemical Formulas C3 to C5 (Comparative Examples 4 to 6) were respectively utilized instead of the compound of Chemical Formula 1a.

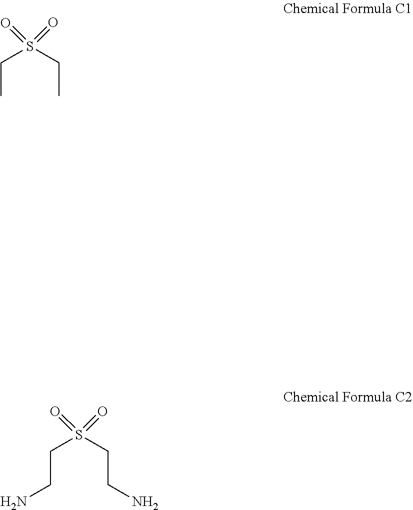

### Examples 4 to 6

A rechargeable lithium battery cell was manufactured in substantially the same manner as in Example 1 except that the compound of Chemical Formula 1a was utilized respectively in an amount of 0.1 parts by weight, 2.0 parts by weight, 5.0 parts by weight.

The additives for an electrolyte according to Examples 1 to 6 and Comparative Examples 1 to 6 had a composition shown in Table 1.

TABLE 1

| | | Other additives | | | | |
|---|---|---|---|---|---|---|
| | | FEC | SN | Solvent | | |
| | Additive | (parts | (parts | (volume | | |
| | (parts by | by | by | ratio) | | |
| | weight) | weight) | weight) | EC | EMC | DMC |
| Example 1 | Chemical | 10 | 0.5 | 20 | 10 | 70 |
| | Formula 1a | | | | | |
| | (0.5) | | | | | |
| Example 2 | Chemical | 10 | 0.5 | 20 | 10 | 70 |
| | Formula 1b | | | | | |
| | (0.5) | | | | | |
| Example 3 | Chemical | 10 | 0.5 | 20 | 10 | 70 |
| | Formula 1c | | | | | |
| | (0.5) | | | | | |
| Example 4 | Chemical | 10 | 0.5 | 20 | 10 | 70 |
| | Formula 1a | | | | | |
| | (0.1) | | | | | |
| Example 5 | Chemical | 10 | 0.5 | 20 | 10 | 70 |
| | Formula 1a | | | | | |
| | (2.0) | | | | | |
| Example 6 | Chemical | 10 | 0.5 | 20 | 10 | 70 |
| | Formula 1a | | | | | |
| | (5.0) | | | | | |
| Comparative | — | 10 | 0.5 | 20 | 10 | 70 |
| Example 1 | | | | | | |
| Comparative | Chemical | 10 | 0.5 | 20 | 10 | 70 |
| Example 2 | Formula C1 | | | | | |
| | (0.5) | | | | | |
| Comparative | Chemical | 10 | 0.5 | 20 | 10 | 70 |
| Example 3 | Formula C2 | | | | | |
| | (0.5) | | | | | |
| Comparative | Chemical | 10 | 0.5 | 20 | 10 | 70 |
| Example 4 | Formula C3 | | | | | |
| | (0.5) | | | | | |
| Comparative | Chemical | 10 | 0.5 | 20 | 10 | 70 |
| Example 5 | Formula C4 | | | | | |
| | (0.5) | | | | | |
| Comparative | Chemical | 10 | 0.5 | 20 | 10 | 70 |
| Example 6 | Formula C5 | | | | | |
| | (0.5) | | | | | |

### Evaluation 1: Evaluation of Room-temperature Charge and Discharge Cycle Characteristics

The rechargeable lithium battery cells according to Examples 1 to 6 and Comparative Examples 1 to 6 were charged and discharged under the following conditions and evaluated with respect to cycle characteristics, and the results are shown in Table 2.

After 200 cycles performing 0.33 C charges (constant current (CC)/constant voltage (CV), 4.3 V, 0.025 C Cut-off)/1.0 C discharges (CC, 2.5 V Cut-off) at 25° C., the cells were evaluated with respect to capacity retention rates and direct current internal resistance (DC-IR) changes.

DC-IR was calculated according to Equations 1 and 2 based on a voltage changed by applying a current of state of charge (SOC) 50 C for 30 seconds, and the results are shown in Table 2.

Capacity retention rate=(Capacity after 200 cycles/Capacity after 1 cycle)*100 Equation 1

DC-IR (direct current internal resistance) variation ratio after 200 cycles={(DC-IR after 200 cycles)/(Initial DC-IR)}*100 Equation 2

TABLE 2

| | Capacity | Initial | DC- | DC-IR variation |
|---|---|---|---|---|
| | retention | DC- | IR after | ratio after 200 |
| | (@25° C., | IR | 200 cycles | cycles |
| | 200 | @25° C. | @25° C. | @25° C. |
| | Cycle) | (mΩ) | (mΩ) | (%) |
| Example 1 | 96.7 | 41.2 | 63.3 | 154 |
| Example 2 | 94.4 | 47.5 | 73.6 | 155 |
| Example 3 | 92.1 | 41.5 | 66.0 | 159 |
| Example 4 | 90.5 | 40.9 | 65.2 | 159 |
| Example 5 | 96.6 | 41.4 | 63.5 | 153 |
| Example 6 | 90.2 | 42.5 | 70.4 | 166 |
| Comparative Example 1 | 53.7 | 42.0 | 147.2 | 350 |
| Comparative Example 2 | 61.8 | 43.8 | 74.5 | 170 |
| Comparative Example 3 | 81.6 | 44.7 | 85.7 | 192 |
| Comparative Example 4 | 86.6 | 43.3 | 74.9 | 173 |
| Comparative Example 5 | 80.8 | 43.5 | 79.2 | 182 |
| Comparative Example 6 | 79.4 | 45.3 | 83.8 | 185 |

Referring to Table 2, when an additive according to the present disclosure was utilized, room temperature cycle-life characteristics were relatively improved.

### Evaluation 2: Evaluation of High-temperature (45° C.) Cycle-Life Characteristics

The rechargeable lithium battery cells according to Examples 1 to 6 and Comparative Examples 1 to 6 were 200 cycles charged and discharged at 45° C. under 0.33 C charge (CC/CV, 4.3V, 0.025 C Cut-off)/1.0 C discharge (CC, 2.5 V Cut-off) conditions and then, measured with respect to capacity retention rates and DC-IR (direct current internal resistance) changes.

Capacity retention and DC-IR was calculated according to Equations 1 and 2 based on a voltage changed by applying a current of SOC 50 C for 30 seconds, and the results are shown in Table 3.

TABLE 3

| | Capacity | Initial | DC-IR | DC-IR variation |
|---|---|---|---|---|
| | retention | DC- | after 200 | ratio |
| | (@45° C., | IR | cycles | after 200 cycles |
| | 200 | @45° C. | @45° C. | @45° C. |
| | Cycles) | (mΩ) | (mΩ) | (%) |
| Example 1 | 95.5 | 40.4 | 71.4 | 177 |
| Example 2 | 92.1 | 44.4 | 81.3 | 183 |
| Example 3 | 89.5 | 42.7 | 76.9 | 180 |
| Example 4 | 91.9 | 40.2 | 98.5 | 245 |
| Example 5 | 95.4 | 40.7 | 73.2 | 180 |
| Example 6 | 86.5 | 42.6 | 110.7 | 260 |
| Comparative Example 1 | 41.2 | 51.4 | 175.4 | 341 |
| Comparative Example 2 | 59.1 | 46.6 | 86.7 | 186 |
| Comparative Example 3 | 77.1 | 46.2 | 89.4 | 194 |
| Comparative Example 4 | 83.7 | 43.3 | 86.0 | 199 |
| Comparative Example 5 | 68.5 | 47.2 | 86.5 | 182 |
| Comparative Example 6 | 70.1 | 45.9 | 84.9 | 185 |

Referring to Table 3, when an additive according to the present disclosure was utilized, high temperature cycle-life characteristics were improved.

### Evaluation 3: ICP-MS Analysis (Ni and Mn Elution Evaluation)

The rechargeable lithium battery cells according to Examples 1 to 6 and Comparative Examples 1 to 6 were 200 cycles charged and discharged under 0.33 C charge (CC/CV, 4.3 V, 0.025 C Cut-off)/1.0 C discharge (CC, 2.5 V Cut-off) conditions at 25° C. and then, measured with respect to Ni and Mn elution amounts in the following method.

The rechargeable lithium battery cells were disassembled to separate positive electrode plates (i.e., positive electrode) therefrom. Subsequently, the separated positive electrode plates were put with an electrolyte in a 10 mL TEFLON (tetrafluoroethylene) container, which was sealed and measured with respect to Ni and Mn contents through an inductively coupled plasma mass spectrometry (ICP-MS) analysis, and the results are shown in Table 4. The electrolyte was a solution in which 1.5 M Li PF₆ was dissolved in a mixed solvent of EC (ethylene carbonate), DEC (diethyl carbonate), and EMC (ethylmethyl carbonate) (a volume ratio of 2:1:7).

TABLE 4

| | Ni detection | Mn detection amount |
|---|---|---|
| | amount (ppm) | (ppm) |
| Example 1 | 311 | 24 |
| Example 2 | 388 | 30 |
| Example 3 | 395 | 35 |
| Example 4 | 402 | 42 |
| Example 5 | 320 | 20 |
| Example 6 | 300 | 18 |
| Comparative Example 1 | 2241 | 278 |
| Comparative Example 2 | 1721 | 141 |
| Comparative Example 3 | 754 | 80 |
| Comparative Example 4 | 459 | 43 |
| Comparative Example 5 | 636 | 92 |
| Comparative Example 6 | 586 | 57 |

Referring to Table 4, the rechargeable lithium battery cells according to Examples 1 to 6 exhibited very low Ni and Mn elution amounts from the electrode plates. However, when the rechargeable lithium battery cells according to Comparative Examples 1 to 6 were compared with the rechargeable lithium battery cells of the Examples, significantly large amounts of Ni and Mn were eluted. Accordingly, in the rechargeable lithium battery cells of the Examples, an amount of gas generated as the cycles progressed was significantly reduced.

### Evaluation 4: Evaluation of High-temperature Storage Characteristics (Capacity Retention Rate/Capacity Recovery Rate/DC-IR Variation Ratio)

The rechargeable lithium battery cells according to Examples 1 to 6 and Comparative Examples 1 to 6 were charged and discharged once at 0.2 C and measured with respect to charge and discharge capacity (before high temperature storage).

In addition, the rechargeable lithium battery cells according to Examples 1 to 6 and Comparative Examples 1 to 6 were charged in or to SOC 100% (a state in which charged to 100% of charge capacity, when total charge capacity was 100%), stored at 60° C. for 30 days, discharged to 3.0 V at 0.2 C under a constant current condition, and then, measured with respect to initial discharge capacity.

In other words, the cells were recharged to 4.3 V under a constant current condition of 0.2 C and at a cut-off current of 0.05 C under a constant voltage condition and discharged to 3.0 V under a constant current condition of 0.2 C and then, measured twice with respect to discharge capacity. A ratio of first discharge capacity to the initial discharge capacity was obtained as capacity retention (retention capacity), and a ratio of second discharge capacity to the initial discharge capacity was obtained as a capacity recovery rate (recovery capacity).

The rechargeable lithium battery cells according to Examples 1 to 6 and Comparative Examples 1 to 6 were measured with respect to ΔV/ΔI (voltage change/current change) as initial DC-IR, and then, DC-IR were measured again by making a maximum energy state inside the cells to a full-charge state (SOC 100%) and storing the cells at a high temperature (60° C.) for 30 days (30D) to calculate a DC-IR increase rate (%) according to Equation 3, and the results are shown in Table 5.

DC-IR increase rate=(DC-IR after 30 days/initial DC-IR)*100 Equation 3

TABLE 5

| | | Capacity | | | |
|---|---|---|---|---|---|
| | Capacity | recovery | Initial | | DC-IR |
| | retention | rate | DC-IR | DC-IR | increase |
| | (@60° C., | (@60° C., | (@25° C., | @60° C., 30D | rate |
| | 30D) | 30D) | (mΩ) | (mΩ) | (%) |
| Example 1 | 97 | 99 | 41.2 | 57.3 | 139 |
| Example 2 | 94 | 96 | 47.5 | 68.9 | 145 |
| Example 3 | 91 | 95 | 43.8 | 65.7 | 150 |
| Example 4 | 75 | 83 | 40.9 | 109.8 | 271 |
| Example 5 | 96 | 99 | 41.4 | 60.0 | 145 |
| Example 6 | 92 | 98 | 42.5 | 70.2 | 164 |
| Comparative Example 1 | 33 | 52 | 42.0 | 215.1 | 511 |
| Comparative Example 2 | 52 | 62 | 43.6 | 100.7 | 231 |
| Comparative Example 3 | 60 | 75 | 44.6 | 97.7 | 219 |
| Comparative Example 4 | 88 | 90 | 43.3 | 69.7 | 161 |
| Comparative Example 5 | 77 | 80 | 45.4 | 84.4 | 186 |
| Comparative Example 6 | 72 | 78 | 47.2 | 83.5 | 177 |

Referring to Table 5, the rechargeable lithium battery cells according to Examples 1 to 6, compared with the rechargeable lithium battery cells according to Comparative Examples 1 to 6, exhibited improved capacity retention rates and capacity recovery rates during the high-temperature storage and were suppressed or reduced from resistance increase rates (i.e., the rates of increase in resistance were suppressed or reduced).

### Evaluation 5: Measurement of Amount of Generated Gas after High-temperature Storage

The rechargeable lithium battery cells according to Examples 1 to 6 and Comparative Examples 1 to 6 were charged to 4.3 V (vs. Li) at 0.1 C under a constant current condition and subsequently, cut off at 0.05 C under a constant voltage mode while maintaining 4.3 V at 25° C. Subsequently, the rechargeable lithium battery cells were disassembled, and positive electrode plates therefrom were put with an electrolyte in a pouch and then, stored in a 60° C. oven for 30 days. A volume change due to a mass change of the pouch was converted in an Archimedes method, and the results are shown in Table 6. The electrolyte was a solution in which 1.5 M LiPF₆ was dissolved in a mixed solvent of EC (ethylene carbonate), DEC (diethyl carbonate), and EMC (ethylmethyl carbonate) (a volume ratio of 2:1:7).

The Archimedes method can be referred to as a method of measuring a weight of the pouch in a water tank filled with water at every specific period (e.g., 4 days) and converting the weight change into a volume change to measure an amount of generated gas.

TABLE 6

| | Amount of generated gas (mL) |
|---|---|
| Example 1 | 0.7 |
| Example 2 | 0.9 |
| Example 3 | 0.9 |
| Example 4 | 1.1 |
| Example 5 | 0.7 |
| Example 6 | 0.8 |
| Comparative Example 1 | 2.8 |
| Comparative Example 2 | 1.6 |
| Comparative Example 3 | 2.2 |
| Comparative Example 4 | 1.2 |
| Comparative Example 5 | 1.3 |
| Comparative Example 6 | 1.3 |

Referring to Table 6, the rechargeable lithium battery cells of Examples 1 to 6 exhibited a reduced amount of generated gas, compared with the rechargeable lithium battery cells of Comparative Examples 1 to 6.

Thus, the rechargeable lithium battery cells manufactured by utilizing an electrolyte including an additive according to the present disclosure exhibited concurrently (e.g., simultaneously) improved room temperature cycle-life and resistance characteristics and also, cycle-life and resistance characteristics when left (e.g., stored) or utilized at a high temperature.

Accordingly, the rechargeable lithium battery cells according to the embodiments of the present disclosure exhibit improved electrolyte impregnation property, excellent or suitable cycle characteristics, reduced resistance after the high-temperature storage, and thus improved high temperature stability.

The terms utilized herein are only utilized to describe specific embodiments and are not intended to limit the present disclosure. Singular expressions may include plural expressions unless the context clearly indicates otherwise. It will be further understood that the terms “comprise”, “include” or “have,” when utilized in the present disclosure, specify the presence of stated features, integers, steps, operations, elements, and/or components, but do not preclude the presence or addition of one or more other features, integers, steps, operations, elements, components, and/or groups thereof. The “/” utilized below may be interpreted as “and” or as “or” depending on the situation.

In the drawings, thickness is enlarged or reduced in order to clearly express one or more layers and regions. Throughout the disclosure, like reference numerals designate like components. Throughout the disclosure, when a component such as a layer, a film, a region, or a plate is mentioned to be placed “on” another component, it will be understood that it may be directly on another component or that another component may be interposed therebetween. Throughout the disclosure, although the terms “first”, “second”, etc., may be utilized herein to describe one or more elements, components, regions, and/or layers, these elements, components, regions, and/or layers should not be limited by these terms. These terms are only utilized to distinguish one component from another component.

The term “ diameter” utilized herein may refer to a particle diameter when the particle is spherical (e.g., substantially spherical), or a major axis length when the particle is non-spherical. The diameter or particle diameter may refer to an average diameter of the particles that may be measured utilizing a particle diameter analyzer (PSA). The “particle diameter” of the particles may be, for example, an average particle diameter. The average particle diameter is the median particle diameter (D50) unless explicitly stated otherwise. The median particle diameter (D50) may refer to a particle diameter corresponding to a cumulative value of 50% calculated from the side of the particle having the smallest particle diameter on the cumulative distribution curve of particle sizes where particles are accumulated in the order of particle diameter from the smallest particle to the largest particle. The cumulative value may be, for example, a cumulative volume. The median particle diameter (D50) may be measured by, for example, laser diffraction.

As utilized herein, the singular forms “a,” “an,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

While the present disclosure has been described in connection with what is presently considered to be practical example embodiments, it is to be understood that the present disclosure is not limited to the disclosed embodiments, but, on the contrary, is intended to cover one or more suitable modifications and equivalent arrangements included within the spirit and scope of the appended claims and equivalents thereof.

### REFERENCE NUMERALS

100: rechargeable lithium battery
112: negative electrode
113: separator
114: positive electrode
120: battery case
140: sealing member

What is claimed is:

1. An additive for an electrolyte, the additive being represented by Chemical Formula 1: 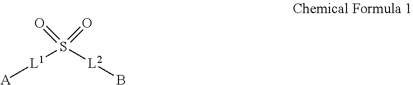
wherein, in Chemical Formula 1,
L¹ and L² are each independently a single bond, a substituted or unsubstituted C1 to C5 alkylene group, a substituted or unsubstituted C2 to C5 alkenylene group, a substituted or unsubstituted C2 to C5 alkynylene group, or a substituted or unsubstituted C6 to C20 arylene group,
A and B are each independently a substituted or unsubstituted C1 to C20 alkyl group, a substituted or unsubstituted C6 to C20 aryl group, or a substituted or unsubstituted C2 to C20 heteroaryl group, and
at least one selected from among A and B is a group represented by Chemical Formula A, 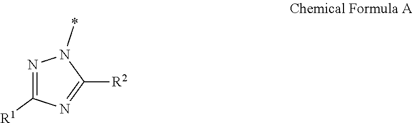
wherein, in Chemical Formula A,
R¹ and R² are each independently hydrogen, a halogen, a substituted or unsubstituted C1 to C10 alkyl group, or a substituted or unsubstituted C3 to C10 cycloalkyl group.

2. The additive of claim 1, wherein
at least one selected from among L¹ and L² is a substituted or unsubstituted C1 to C5 alkylene group.

3. The additive of claim 1, wherein
L¹ and L² are each independently a substituted or unsubstituted C1 to C5 alkylene group.

4. The additive of claim 1, wherein
at least one selected from among L¹ and L² is a substituted or unsubstituted C2 to C5 alkylene group.

5. The additive of claim 1, wherein
L¹ and L² are each independently a substituted or unsubstituted C2 to C5 alkylene group.

6. The additive of claim 1, wherein
Chemical Formula 1 is represented by Chemical Formula 1-1: 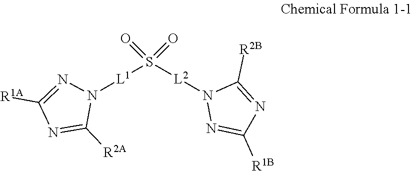
wherein, in Chemical Formula 1-1,
L¹ and L² are each independently a substituted or unsubstituted C2 to C5 alkylene group, and
R^{1A}, R^{1B}, R^{2A} and R^{2B} are each independently hydrogen, a halogen, a substituted or unsubstituted C1 to C10 alkyl group, or a substituted or unsubstituted C3 to C10 cycloalkyl group.

7. The additive of claim 1, wherein
the additive is selected from the compounds of Group 1:

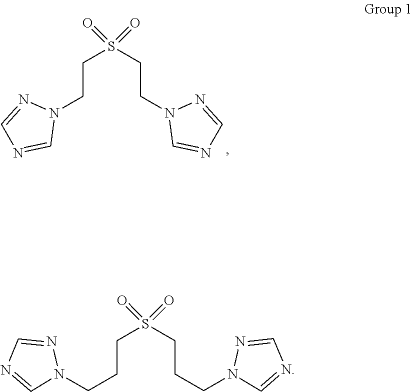 [SMILES: O=S(=O)(CCn1cncn1)CCn1cncn1 ; O=S(=O)(CCCn1cncn1)CCCn1cncn1]

8. An electrolyte for a rechargeable lithium battery, the electrolyte comprising
a non-aqueous organic solvent, a lithium salt, and the additive of claim 1.

9. The electrolyte of claim 8, wherein
the additive is in an amount of about 0.01 to 5.0 parts by weight based on 100 parts by weight of the electrolyte.

10. The electrolyte of claim 8, wherein
the additive is included in an amount of about 0.05 to 3.0 parts by weight based on 100 parts by weight of the electrolyte.

11. The electrolyte of claim 8, wherein
the electrolyte further comprises at least one other additive selected from among vinylene carbonate (VC), fluoroethylene carbonate (FEC), difluoroethylene carbonate, chloroethylene carbonate, dichloroethylene carbonate, bromoethylene carbonate, dibromoethylene carbonate, nitroethylene carbonate, cyanoethylene carbonate, vinylethylene carbonate (VEC), adiponitrile (AN), succinonitrile (SN), 1,3,6-hexane tricyanide (HTCN), propenesultone (PST), propanesultone (PS), lithium tetrafluoroborate (LiBF₄), lithium difluorophosphate (LiPO₂F₂), and 2-fluoro biphenyl (2-FBP).

12. A rechargeable lithium battery, comprising
a positive electrode comprising a positive active material;
a negative electrode comprising a negative active material; and
the electrolyte of claim 8.

13. The rechargeable lithium battery of claim 12, wherein
the negative active material comprises at least one selected from among graphite and a Si composite.

14. The rechargeable lithium battery of claim 13, wherein
the Si composite comprises a core comprising Si-based particles and an amorphous carbon coating layer.

15. The rechargeable lithium battery of claim 14, wherein
the Si-based particles comprise at least one selected from among Si particles, a Si—C composite, SiOₓ (0<x≤2), and a Si alloy.

16. The rechargeable lithium battery of claim 12, wherein
the rechargeable lithium battery operates at a high voltage of greater than or equal to about 4.3 V.

17. The electrolyte of claim 8, wherein
Chemical Formula 1 is represented by Chemical Formula 1-1: 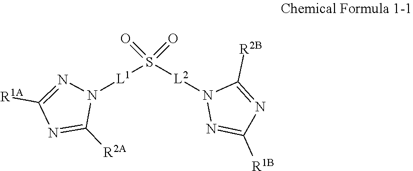
wherein, in Chemical Formula 1-1,
L¹ and L² are each independently a substituted or unsubstituted C2 to C5 alkylene group, and
R^{1A}, R^{1B}, R^{2A} and R^{2B} are each independently hydrogen, a halogen, a substituted or unsubstituted C1 to C10 alkyl group, or a substituted or unsubstituted C3 to C10 cycloalkyl group.

18. The electrolyte of claim 8, wherein
the additive is selected from the compounds of Group 1:

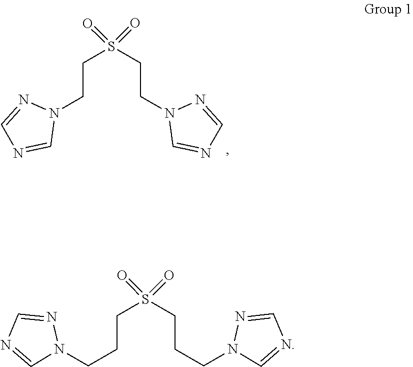 [SMILES: O=S(=O)(CCn1cncn1)CCn1cncn1 ; O=S(=O)(CCCn1cncn1)CCCn1cncn1]

19. The rechargeable lithium battery of claim 14, wherein
Chemical Formula 1 is represented by Chemical Formula 1-1: 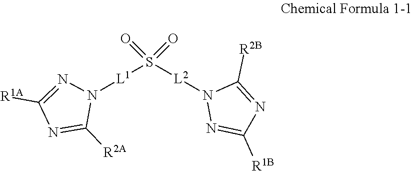
wherein, in Chemical Formula 1-1,
L¹ and L² are each independently a substituted or unsubstituted C2 to C5 alkylene group, and
R^{1A}, R^{1B}, R^{2A} and R^{2B} are each independently hydrogen, a halogen, a substituted or unsubstituted C1 to C10 alkyl group, or a substituted or unsubstituted C3 to C10 cycloalkyl group.

20. The rechargeable lithium battery of claim 14, wherein the additive is selected from the compounds of Group 1: 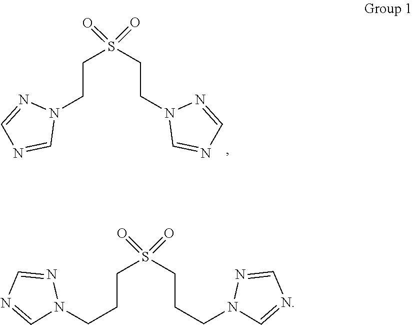 [SMILES: O=S(=O)(CCn1cncn1)CCn1cncn1 ; O=S(=O)(CCCn1cncn1)CCCn1cncn1]
